(19) United States
(12) Patent Application Publication
(10) Pub. No.: US 2019/0366068 A1
(43) Pub. Date: Dec. 5, 2019

(54) MICRONEEDLE FOR BIOSENSING AND METHOD OF FABRICATION

(71) Applicant: Zibo Tanwei Nanotechnology Co., Ltd., Zibo Shangdong Province (CN)

(72) Inventors: Yan Li, Waterloo (CA); Hang Zhang, Waterloo (CA); Eric J. M. Blondeel, Waterloo (CA); Bo Cui, Waterloo (CA)

(21) Appl. No.: 16/415,628

(22) Filed: May 17, 2019

Related U.S. Application Data
(60) Provisional application No. 62/677,521, filed on May 29, 2018.

(51) Int. Cl. A61M 37/00; G03F 7/00; A61B 5/00

(52) U.S. Cl. CPC A61M 37/0015; A61B 5/1473; A61B 5/685; G03F 7/0035

(57) ABSTRACT

A method of fabricating a sharpened microneedle using in-plane manufacturing techniques. The method includes depositing at least one layer of photoresist on a broad surface of a substrate, and the photoresist is subsequently patterned to define features of the microneedle. A first etching treatment in the form of deep reactive ion etching (DRIE) is applied to etch the substrate into first structure where the etching depth is less than the substrate total depth so as to create at least one sacrificial feature adjacent to the portions of the substrate under the photoresist. A second etching treatment in the form of isotropic wet etching is subsequent applied where the etching of the at least one sacrificial feature causes a gradual variation in etching rate along the length of the microneedle such that the tip of the microneedle is sharpened.

### CROSS-REFERENCE TO RELATED APPLICATION(S)

This application claims the benefit of priority to U.S. Provisional Patent Application No. 62/677,521 entitled “MICRONEEDLE FOR BIOSENSING AND METHOD OF FABRICATION” and filed on May 29, 2018, the contents of which are incorporated herein by reference.

### TECHNICAL FIELD

This invention relates generally to the medical device field, and more specifically to a new and useful microneedle device for biosensing in the medical device field.

### BACKGROUND

Biosensing devices are commonly used in the medical device field to sample one or more fluids from a user for the purposes of testing and monitoring body chemistry. Microneedles and microneedle arrays have shown promise in this field, as they can be inserted into skin of a user in a relatively easy and pain-free manner, often by the user himself in a home setting.

Current manufacturing methods and materials, however, have limitations with respect to creating effective microneedles and microneedle arrays. In out-of-plane manufacturing methods with semiconductor materials like silicon, for instance, the microneedle length is limited by the anisotropic Bosch etching process, as well as the thickness of the substrate wafer used to fabricate the microneedle and/or limited by the structural integrity of out-of-plane structures, which can limit the type and amount of fluid that the microneedle is able to access within the user. While in-plane manufacturing techniques can result in relatively long microneedle lengths, the resulting needles are often not sharp enough to overcome skin elasticity during device insertion, or, if they are able to overcome skin elasticity, the needle tips (e.g., wedge-shaped tips) often cut through skin like a scalpel, which induces bleeding and prevents collection of interstitial fluid.

Thus, there is a need in the medical device field to create a new and useful method for manufacturing a microneedle device for biosensing.

### SUMMARY OF THE INVENTION

In accordance with one example embodiment, there is provided a method of fabricating a needle, the method comprising: providing a semiconductor substrate; applying a first photoresist to a first surface of the substrate; patterning the first photoresist to define a first etching pattern; performing a first etching treatment to the substrate, the first etching pattern causing the substrate to be etched into a first structure, the first structure including at least one sacrificial feature; and performing a second etching treatment on the first structure to etch the first structure into a second structure, wherein the at least one sacrificial feature is at least partially etched away during the second etching treatment causing one or more features of the needle to be etched into the second structure.

In any of the above, the method may be an in-plane method of fabricating the needle.

Any of the above may further comprise pre-treating the substrate, which includes baking the substrate, cleaning the substrate, creating a clean and dry first surface, enhancing adhesion with the first photoresist, cleaning the substrate by immersion in hydrofluoric acid, cleaning by a solvent, rinsing by deionized water, or blow drying.

Any of the above may further comprise one or more of removing the first photoresist, curing the first photoresist, assembling multiple needles together, adding a biocompatible material to the needle, or adding a wicking material.

In any of the above, the applying may include applying a single layer of the first photoresist, or a double layer of the first photoresist.

In any of the above, the patterning may include defining a channel opening with a width in the first photoresist, the first etching treatment including etching a channel into the first structure by aspect ratio dependent etching (ARDE), a depth of the channel is defined by the width of the channel opening.

In any of the above, the at least partial etching of the at least one sacrificial feature during the second etching treatment may cause the one or more needle feature to include a sharpened needle tip.

In any of the above, the one or more features of the needle may include the shape of a needle end face, a length of the needle, and an overall diameter of the needle.

In any of the above, the first etching treatment may comprise deep reactive ion etching (DRIE).

In any of the above, the first etching treatment may be performed so that portions of the substrate adjacent to the patterned first photoresist is etched to a depth less than a total depth of the substrate thereby defining the at least one sacrificial feature.

In any of the above, the one or more features of the needle may include at least one needle cross section, etching away the at least one sacrificial feature during the second etching treatment causes a variation in an etching rate along a lengthwise direction of the at least one needle cross section such that a first end of the at least one needle cross section is etched into a sharpened needle tip.

In any of the above, the second etching treatment may comprise isotropic wet etching.

In any of the above, the second etching treatment may further comprise removing portions of the first structure to a variable depth thereby creating a sloped profile.

In any of the above, the substrate may be a low resistivity, p-type, single side-polished silicon wafer with a predetermined crystal orientation.

In any of the above, a length of the needle may be between 1 mm to 2 mm.

Any of the above may further comprise: applying a second photoresist to a second surface of the substrate, patterning the second photoresist to define a second etching pattern, the second etching pattern defines at least one fluidic channel of the needle, performing a third etching treatment to the substrate, the second etching pattern causing the at least one fluidic channel to be etched into the second surface of the substrate; attaching an additional sacrificial feature to the second surface of the second structure such that the at least one fluidic channel is covered by the additional sacrificial feature; and wherein the additional sacrificial feature that is at least partially etched away during the second etching treatment minimizes or eliminates any etching to the at least one fluidic channel during the second etching treatment.

Any of the above may further comprise removing the second photoresist after the third etching treatment.

In any of the above, the at least one fluidic channel may define a microfluidic network.

In any of the above, the third etching treatment may comprise DRIE.

In any of the above, the additional sacrificial feature may comprise a silicon wafer.

In any of the above, the additional sacrificial feature may be attached to the second surface of the substrate with tape comprising a polyimide film with a silicon adhesive.

In any of the above, the at least one channel may extend through the needle tip and positioned away from an apex of the tip.

In any of the above, the at least one channel may extend through the tip offset at an axis away from a longitudinal axis defined by the tip

Any of the above may further comprise: applying a second photoresist to a second surface of the substrate, patterning the second photoresist to define a second etching pattern, the second etching pattern defines at least one fluidic channel of the needle, and performing a third etching treatment to the substrate, the second etching pattern causing the at least one fluidic channel to be etched into the second surface of the substrate.

In any of the above, the needle may be a microneedle, wherein the tip has a radius within micron range.

In any of the above, the tip may have a radius less than or greater than micron range.

A needle for reaching and sampling interstitial fluid of a user produced by any of the above methods.

In accordance with another example embodiment is a microneedle produced by the described methods.

### BRIEF DESCRIPTION OF THE FIGURES

FIG. 1 is a schematic representation of the microneedle device.

FIG. 2 is a workflow of a method for manufacturing a microneedle.

FIG. 3 is a variation of a method for manufacturing a microneedle.

FIG. 4 is a variation of a method for manufacturing a microneedle.

FIG. 5 is a variation of a method for manufacturing a microneedle.

FIGS. 6A and 6B depict an embodiment of a microneedle device created by a variation of a method for manufacturing a microneedle.

FIG. 7 depicts an embodiment of a microneedle device.

FIGS. 8A-8G depict an embodiment of a microneedle device.

FIG. 9 depicts an embodiment of a microneedle device created by a variation of a method for manufacturing a microneedle.

FIGS. 10(a)-10(c) depict undesired etching of the channel using the variation of the method depicted in FIG. 2.

FIGS. 10(d)-10(i) depict a variation of a method for manufacturing a microneedle that minimizes channel etching.

### DESCRIPTION OF THE EXAMPLE EMBODIMENTS

The following description of the example embodiments of the invention is not intended to limit the invention to these example embodiments, but rather to enable any person skilled in the art to make and use this invention.

### 1. Overview

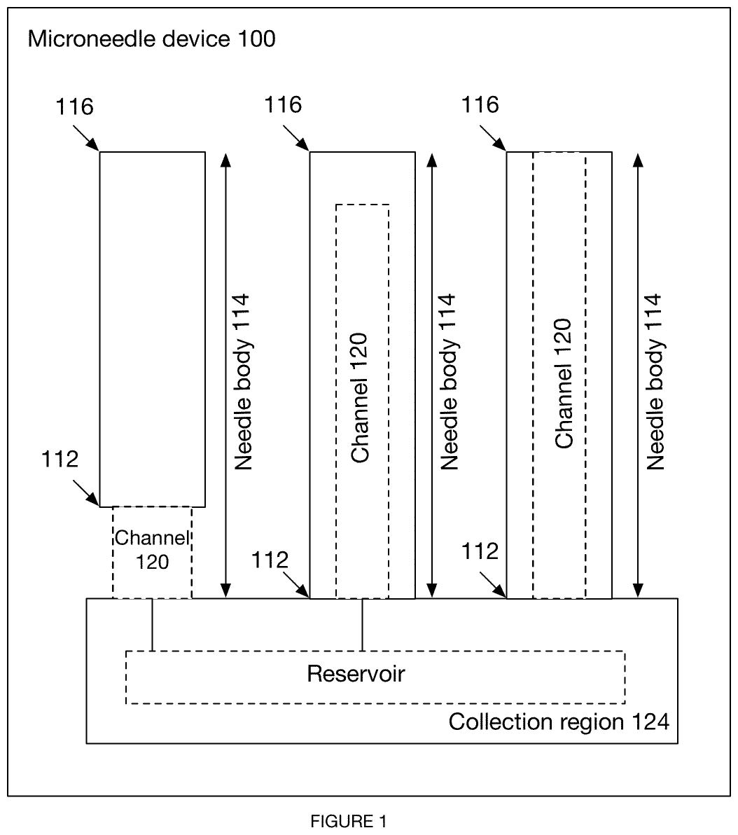

As shown in FIG. 1, an embodiment of a microneedle device includes an array of microneedles, each microneedle defining a base, body, and tip, and a set of channels arranged at least partially within the array of microneedles. Additionally or alternatively, the microneedle array can include any number of microneedles, and any number of channels, arranged in any suitable configuration, with a reservoir fluidly connected to one or more channels, or any other suitable component and arrangement of components.

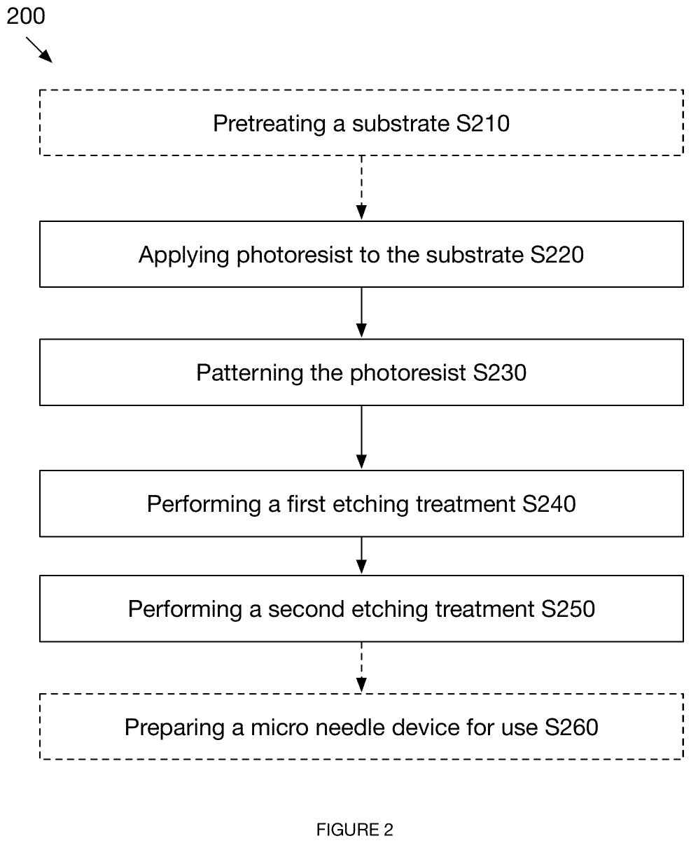

As shown in FIG. 2, an embodiment of method 200 for manufacturing a microneedle device includes applying photoresist to a substrate S220, selectively exposing the substrate to light S230, performing a first etching treatment S240, and performing a second etching treatment S250. Additionally or alternatively, the method 200 can include pre-treating the substrate S210, applying a post-etching treatment to the substrate S260, or any other step or combination of steps performed in any suitable order.

### 2. Benefits

The method for manufacturing a microneedle device and the resulting microneedle device can confer several benefits over current manufacturing methods and microneedles.

First, in some variations, the microneedle length is manufactured in-plane, wherein an in-plane projection of the needle represents a cross section along a frontal or medial plane of the needle. In this manufacturing setup, the needle length is not limited by the Bosch etching process, or the thickness of the substrate, as is the case in out-of-plane manufacturing where the needle length is constructed along the substrate thickness (in-plane projection represents a cross section along a transverse plane of the needle). In-plane manufacturing allows for longer needles to be manufactured, which can consequently sample fluids (e.g., interstitial fluid) from relatively deep depths below the skin surface of the user.

Second, in some variations, the microneedle device is manufactured, at least in part, from isotropic etching techniques. This allows for sharpening of the tips of needles manufactured in-plane. Since sharper needles better penetrate through skin, which typically has a high elasticity, this can allow for easier and more painless device insertion. It has been know that existing microneedle manufacturing methods typically produce microneedles with a wedge shaped end, as opposed to a tapered tip. Although such needles may penetrate through brain tissues, which are softer than skin tissues, they are undesirable for skin tissue applications as the needles may cause pain and tissue damage. Needles with sharpened tips as manufactured in accordance with the present disclosure may minimize skin tissue damage and hence more advantageous at least in neural probe applications.

In variants, this can be accomplished by leveraging temporary substrate features (e.g., sacrificial walls that control which portions of the microneedle are etched away in the isotropic etching process (e.g., by leveraging the mass transport properties of the etchant and substrate). For example, primary areas adjacent the sacrificial walls can be etched slower than secondary areas distal the sacrificial walls, which can result in a tapered profile extending from the primary areas (forming the needle tip) to the secondary areas (proximal the needle base).

In other variants, this can be accomplished by applying a secondary mask or by leaving the photoresist on during the isotropic etching process, wherein the mask functions to prevent the substrate contiguous the mask from being etched. This can result in a tapered profile extending from the substrate face proximal the mask (forming the needle tip) to a substrate face distal the mask (proximal the needle base). In one set of variants, for instance, “bottom-up” etching (e.g., dry etching) is performed with a layer of photoresist to generate the sharp microneedle tip.

However, the in-plane needle can be otherwise sharpened. In one set of variants, for instance, “top-down” etching (e.g., wet etching) is performed without a layer of photoresist to generate the sharp microneedle tip.

Additionally or alternatively, the methods and systems described below can confer any other suitable benefit over conventional manufacturing methods and microneedle devices.

### 3. System—Microneedle Device

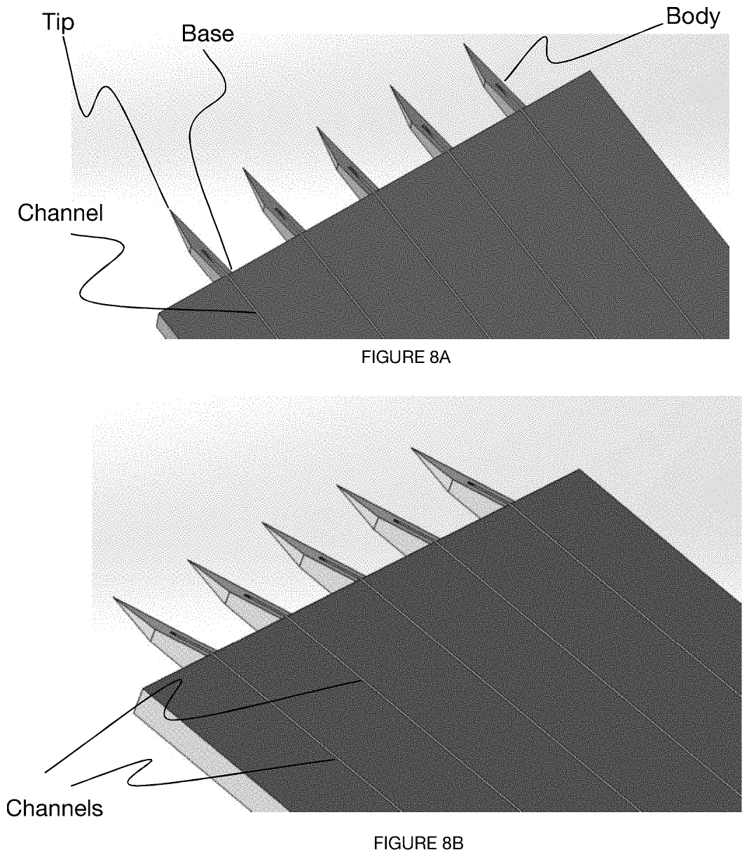

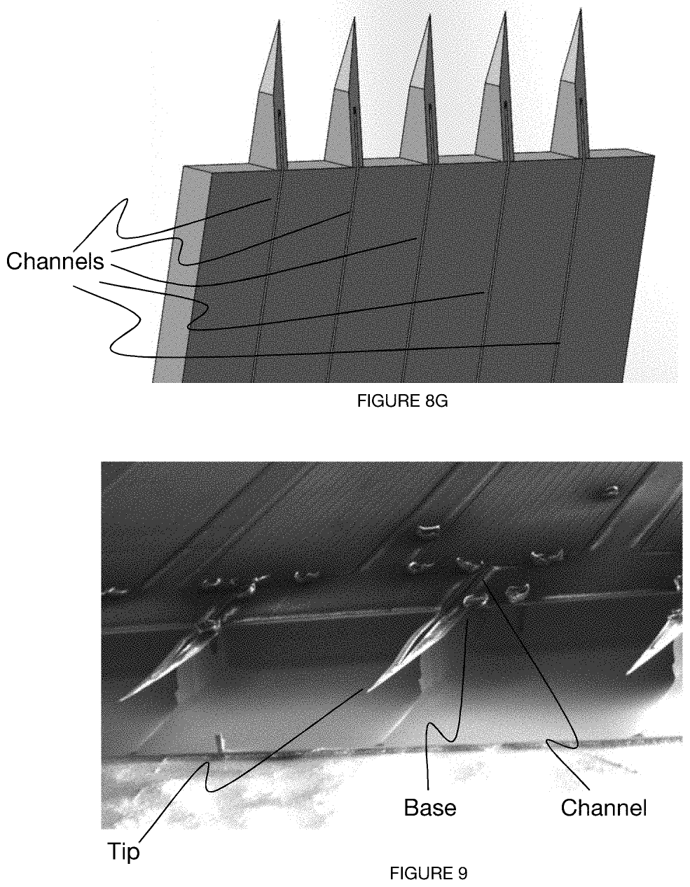

The microneedle device includes an array of microneedles, each microneedle defining a base, body, and tip, and a set of channels arranged at least partially within the array of microneedles. Additionally or alternatively, the microneedle array can include a single microneedle, any number of channels arranged in any suitable configuration, a reservoir fluidly connected to one or more channels, or any other suitable component and arrangement of components. One embodiment of the microneedle device 100 is shown in FIGS. 6A and 6B. Another embodiment of the microneedle device 100 is shown in FIG. 9.

The microneedle device 100 preferably includes an array of microneedles 110. Preferably, the array of microneedles includes multiple microneedles arranged in a row, but can additionally or alternatively include a single microneedle or multiple microneedles arranged in any suitable configuration. As shown in FIG. 1, each microneedle includes a body 114, each body 114 defining a base 112 at a distal end and a tip 116 opposing the base 112 at a proximal end of the needle body 114.

The body 114 can have any suitable geometry, such as those produced by any variation or combination of variations of the method 200 described below. In exemplary variations, the microneedle body 114 includes or is in the form of a prism (e.g., right prism, oblique prism, etc.), wherein the prism is truncated, the truncation of the prism contributing to a sharpness of the microneedle tip. With the truncation, the two end faces of the prism can have different numbers of sides with respect to each other. The truncation can be planar, concave, or convex. However, the body 114 can have any suitable configuration or geometry.

In one variation, one end face of a prism can have a smaller number of sides than an opposing end face. In a specific example, for instance, a truncated pentagonal prism can have five sides on a first end face and four sides on a second end face, such as in the case of a truncation plane intersecting only with the second end face. In another specific example, a truncated pentagonal prism can have five sides on a first end face and six sides on an opposing second end face, such as in the case of a truncation plane which intersects only with the second end face. In other examples, the truncation plane can intersect with both end faces, creating any number of sides for the first and second end faces (e.g., same number of sides, different number of sides, two greater sides for a first end face than a second end face, etc.). In a specific example of this, for instance, a truncation plane intersecting with both end faces, each end face part of a pentagonal prism, can result in a first end face having six sides and a second end face having four sides.

Additionally or alternatively, the needle body can be formed from any other kind of structure, truncated or not, such as any kind of prism (e.g., triangular prism, pentagonal prism, etc.), cylinder, pyramid, polyhedral structure, or any other structure having any suitable geometry (e.g., number of faces, number of sides per face, dimensions, etc.).

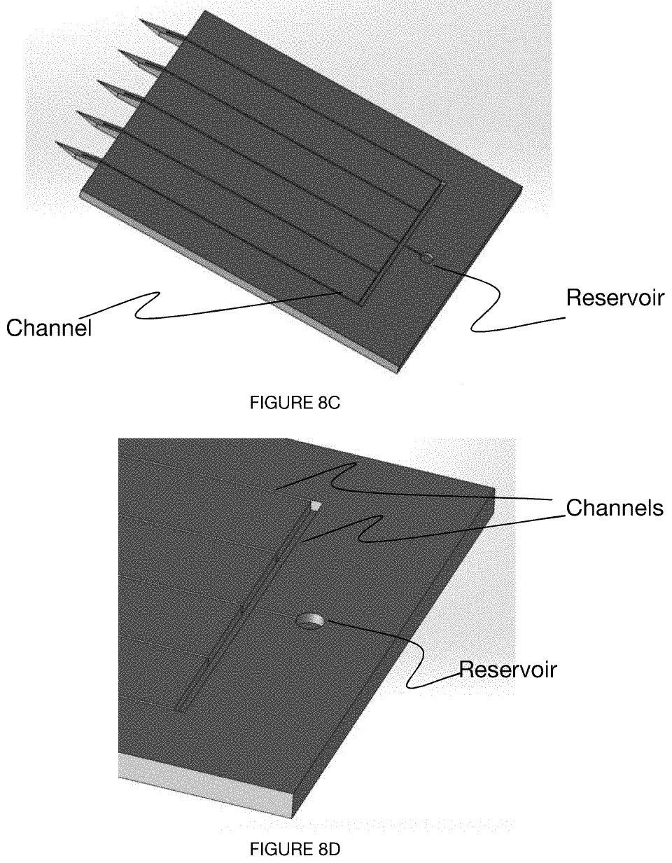

As shown in FIG. 7, the microneedle body 114 can be in the form of a truncated quadrilateral prism, the quadrilateral prism having a first broad end face and an opposing second broad end face, each of the broad end faces aligned with a broad surface of the substrate. Preferably one of the broad end faces intersects with a truncation plane, wherein the truncation plane is oriented at a nonzero angle with respect to a broad end face, but additionally or alternatively, both broad end faces can intersect with the truncation plane, no broad faces intersect with a truncation plane, the prism intersects with multiple truncation planes, or any other suitable portion of the prism can be truncated in any suitable way.

The body 114 of the microneedle can be solid, hollow, indented, or have any areas of removed material or negative space (e.g., needle having a channel running down broad surface of body, needle having a channel bisecting the needle tip, etc.). The length of the microneedle is preferably configured to reach and sample interstitial fluid of a user (e.g., penetrate within or past the stratum corneum, the epidermis, the dermis, the reticular dermis, etc.), but can additionally or alternatively be configured to reach blood of a user, or any other suitable fluid or tissue of a user at any suitable depth. The needle can have a length of 1.5 millimeters (mm), between 1 mm and 2 mm, between 2 mm and 4 mm, smaller than 1 mm, in excess of 4 mm, or any other suitable length or ranges of length.

The tip 116 of the needle preferably has a small enough cross-sectional dimension (e.g., radius, diameter, etc.) to penetrate skin of the user (e.g., overcome a skin elasticity threshold, overcome a skin elasticity threshold with an insertion force below a predetermined force threshold) without breaking off during or after insertion. The tip preferably tapers to a radius of approximately 10 microns, but can alternatively taper to an infinitesimally small radius, a radius of approximately 5 microns, 15 microns, 20 microns, between 5 and 15 microns, below 5 microns, above 10 microns, or any other suitable radius or range of radii. In some variations, the needle tip is split into two or more tips, such as in the case of a microneedle having a channel running all the way along (e.g., bisecting) a tip region. In these variations, the tip can be completely bisected, if the channel depth is equal to the needle thickness, The tip can be aligned with an axis (e.g., centerline) of the microneedle body, but can additionally or alternatively be located along a face (e.g., along an end face of a truncated prism) of the needle body, a side (e.g., along a side of a side face of a truncated prism, along a side of an end face of a prism, etc.) of the needle body, along both sides of a needle body (e.g., bisected tip), or have any other suitable location with respect to the microneedle body.

The microneedle device is preferably fully but can alternatively be partially constructed (e.g., through etching) from a substrate 118. The substrate 118 preferably includes silicon (e.g., silicon having a purity of 99.9999999%, a purity greater than 99%, a purity between 98% and 100%, a purity less than 98%, etc.), further preferably a low-resistivity p-type silicon. Additionally or alternatively, the silicon can be an n-type silicon, a relatively resistive silicon, have any suitable purity, or any other properties. Further additionally or alternatively, the wafer can include any number of semiconductor materials, non-metallic materials (e.g., glass, wood, paper, fabric, etc.), semi-metallic materials, metallic materials (e.g., aluminum, gold, etc.), wicking materials (e.g., paper, fabric, etc.), or any other suitable material or combination of materials. The substrate material can be pure, doped (e.g., with boron, aluminum, phosphorus, nitrogen, gold, etc.), or otherwise combined with any suitable material. The substrate can have any suitable surface properties (e.g., polished, coated, etc.), such as one or more polished surfaces. In example variations, the substrate 118 is a low resistivity, p-type, single-side-polished silicon wafer with a predetermined crystal orientation (e.g., (100) crystal orientation). In other variations, the substrate 118 includes any material(s) configured for etching, such as a metallic material.

The shape (e.g., circular, rectangular, etc.) and dimensions of the wafer are preferably determined based on desired features of the microneedle device (e.g., microneedle length, number of microneedles, depth of channels, etc.), but can additionally or alternatively be chosen based on industry standards, manufacturing standards, or any other parameters. The thickness (e.g., along z-dimension) is preferably determined based on a maximum aspect ratio of etching achievable with subsequent etching steps (e.g., thickness greater than maximum possible etching depth), such that channels and reservoirs are not etched throughout the entire substrate thickness, but can additionally or alternatively be determined based on any other parameter(s). In one variation, the silicon wafer is circular with a 4-inch diameter, and a thickness (e.g., dimension along z-direction) between 300 and 500 microns.

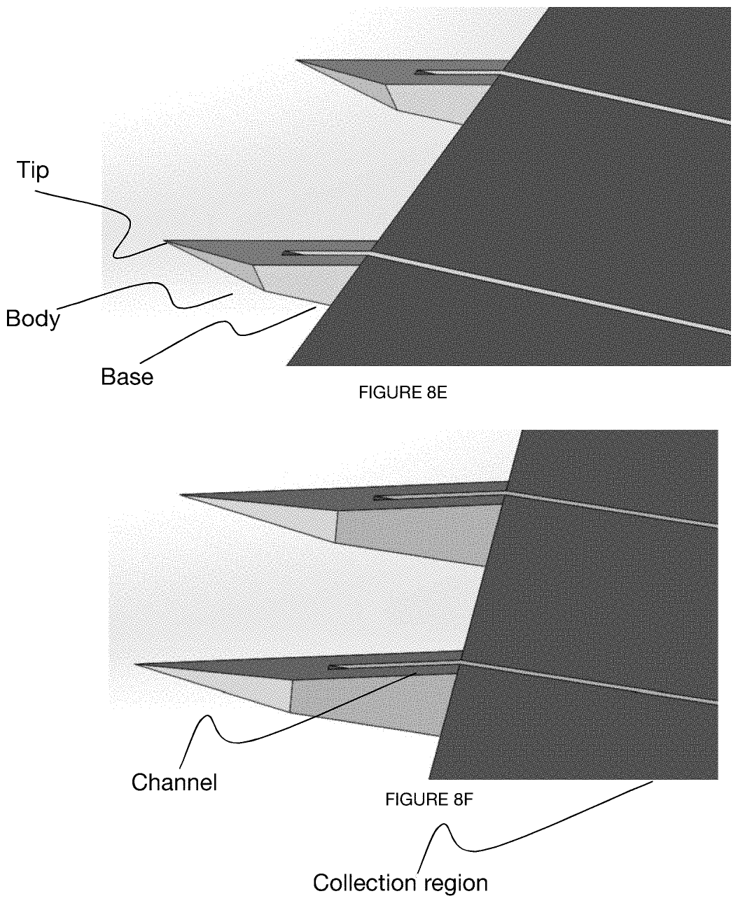

In some variations, the microneedle device includes a set of one or more channels 120, wherein the set of channels 120 functions to transport one or more fluids out of a user. In some variations, the fluids can further be transmitted to a collection region 124 of the microneedle device, the collection region 124 fluidly connected to one or more needle bases. The collection region 124 is preferably arranged distal to a set of microneedle bases but can additionally or alternatively be defined adjacent to a microneedle base, arranged distal to a single microneedle base, or otherwise arranged. The set of channels 120 can be partially enclosed within the microneedle device (e.g., three-walled channels), fully enclosed, or not enclosed at all (e.g., when a surface of the microneedle device enables sufficient capillary action). The set of one or more channels 120 can be distributed in any arrangement with the set of microneedles: there can be a single channel 120 for each microneedle 110, multiple channels for each microneedle 110, one channel shared among multiple microneedles, or any other suitable distribution and arrangement of channels. In variations of the microneedle device having multiple channels 120, the channels 120 can be isolated (e.g., for independent microneedle testing), fluidly connected (e.g., converging in a reservoir, as shown in FIGS. 8C-8D), or otherwise arranged.

Each channel in a set of channels can have a uniform width (e.g., 500 microns, 1 mm, between 100 microns and 1 mm, less than 100 microns, greater than 1 mm, etc.) along its entire length, a varying width along its length, a different width in comparison to a width of another channel in the set of channels, the same width in comparison to a width of another channel in the set of channels, or any suitable width. The dimensions of the channel can be determined in part or in full based on principles of aspect-ratio-dependent etching, wherein small features etch slower than large features. In one variation, the width of one or more channels is determined based on a dimension of another negative space feature (e.g., spacing between needles, reservoir, etc.), such that the channel does not etch as fast as the other negative space feature.

The channels can define a straight path, curved path, sinuous path, random path, or any other suitable path. Each of the channels preferably has a depth smaller than the thickness of the substrate but can alternatively pass all the way through the thickness of the substrate along a partial or full length of the needle body, such as in some variations having a wicking material arranged within part or all of the channel.

The microneedle device can include a set of channels (e.g., as shown in FIG. 7, as shown in FIGS. 8A-8G) arranged in at least a portion of each of the microneedles. In a specific example, each of the set of channels can be defined by three walls of a notch in a broad surface (e.g., in-plane) of the microneedle device, wherein fluid from a user travels in a direction from tip to base through capillary action enabled by the three walls of the channel. The channels can converge into a shared reservoir or remain separate.

In one variation, the microneedle device includes a channel which extends along the body of a microneedle, covering an entire length of the microneedle from tip to base. Alternatively, the channel can extend for a partial length of the microneedle between the tip and base.

In another variation, the microneedle device includes a channel which extends from the base in a direction away from the tip.

In yet another variation, the microneedle device can have a main channel (e.g., relatively large width) distal to each of the bases of a set of microneedles, wherein the main channel is fluidly connected to a set of microchannels, each of the microchannels arranged along the body of each of the set of microneedles. In a specific example, the main channel is arranged perpendicular to each of the set of microchannels. In other examples, the main channel and microchannels can be arranged in any suitable way.

The microneedle device can include any number of reservoirs 122 arranged in a collection region 124 of the microneedle device, preferably fluidly connected to each of a set of channels but additionally or alternatively can be fluidly isolated from one or more channels, fluidly connected to a wicking material, or otherwise arranged. The reservoir(s) 122 can be accessible to a supplementary component (e.g., test strip), coupled to a collection device (e.g., collection vial), closed to the environment, open to the environment, or configured in any other suitable way. The reservoir preferably has the same depth, as measured perpendicular to a broad surface of the substrate, as one or more channels, but can alternatively have a different depth, a substantially nonzero depth (e.g., fluid collects on surface of microneedle device), or any other suitable depth. In one variation, the microneedle device includes a single reservoir 122 fluidly connected to multiple channels 120. In another variation, each channel includes its own reservoir. In yet another variation, each channel functions as a reservoir. Additionally or alternatively, the microneedle device can include any other suitable number and arrangement of reservoirs in relation to any number of channels.

### 4. Method—Overview

A method 200 for manufacturing a microneedle device can include applying photoresist to a substrate S220, selectively exposing the substrate to light S230, performing a first etching treatment S240, and performing a second etching treatment S250. Additionally, the method 200 can include pre-treating a substrate S210, applying a supplementary treatment S260, or any other suitable step or combination of steps. The method preferably produces any or all of the microneedle device 100 described previously, but can additionally or alternatively produce any suitable microneedle device 100.

### 4.1 Method—Applying Photoresist to a Substrate S220

The method 200 can include applying photoresist to a substrate S220, which functions to enable a prescribed pattern of etching, thereby creating a predetermined set of microneedle features (e.g., length, radius, shape, etc.). Additionally or alternatively, S220 can function to create one or more microneedle device features (e.g., through the application of curable, un-strippable photoresist).

S220 is preferably performed first in the method 200, but can additionally or alternatively be performed multiple times throughout the method 200 (e.g., prior to each of a set etching steps), or at any other point in the method 200.

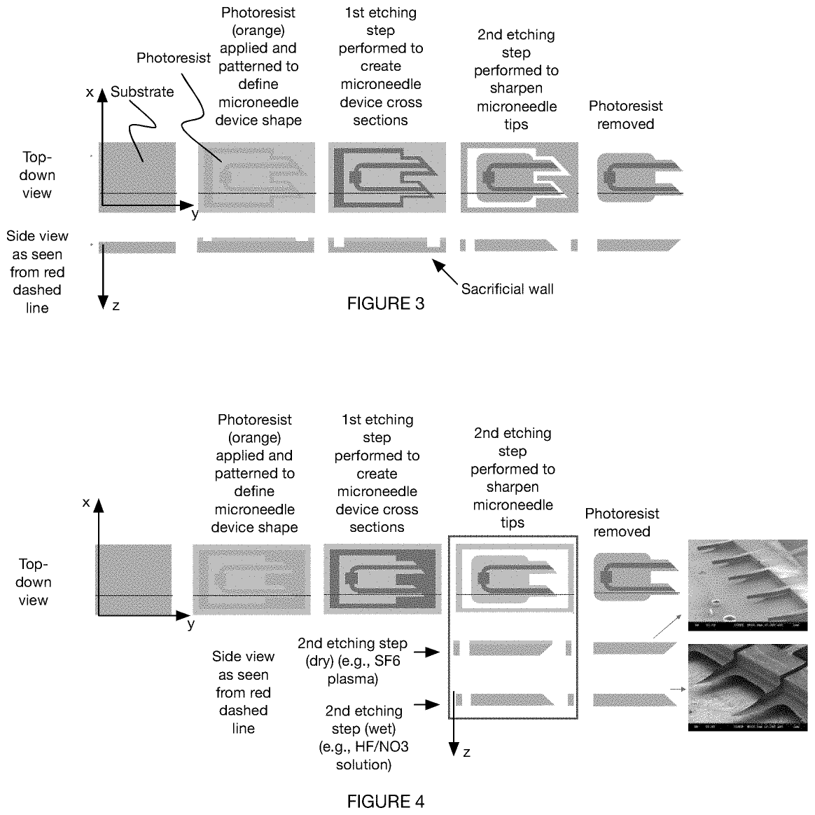

As shown in FIG. 3, the photoresist is preferably applied to a substrate (e.g., silicon wafer) at a broad surface of the substrate (e.g., superior broad surface, polished surface, etc.), but can additionally or alternatively be applied on multiple surfaces of a substrate (e.g., broad surface and side surfaces), all surfaces of the substrate, a partial surface of the substrate, or any other region or surface of a substrate. The photoresist can be applied through any or all of: immersion (e.g., dip-coating), spraying, spin-coating (e.g., at 200 revolutions per minute for 40 seconds), application of a dry film photoresist, painting, or any other suitable application process. A positive photoresist (e.g., photopolymeric photoresist, photocrosslinking photoresist, etc.), negative photoresist (e.g., photodecomposing photoresist), permanent photoresist (e.g., un-strippable, curable, etc.), temporary photoresist (e.g., strippable, dissolvable, etc.), or any other suitable photoresist or combination of photoresists can be applied to the substrate. The photoresist preferably has a low or negligible etching rate relative to the etching rate of the substrate material, but can alternatively have any suitable etching rate.

In one variation, a positive photoresist is used, wherein the corresponding mask includes the needle features (e.g., needle cross section). In a specific example, AZ 4620 (Microchemicals) positive photoresist is used. Alternatively, a negative photoresist can be used, wherein the corresponding mask includes cross sections of channels and reservoirs.

The photoresist can be applied to any suitable thickness (e.g., 11 microns, 24 microns, greater than 11 microns, between 5 and 30 microns, etc.) and with any number of layers (e.g., single layer, double layer with pre-baking between layers, etc.). In one variation, a double layered photoresist (e.g., 24 microns thick) is applied.

Step S220 can additionally include any suitable sub-processes, such as, but not limited to: curing a photoresist, drying a substrate having applied photoresist, baking a substrate (e.g., pre-baking at 90 degrees Celsius for 5 minutes), or any other suitable sub-processes.

### 4.2 Method—Patterning the Photoresist S230

The method 200 can include patterning the photoresist S230, which functions to pattern a substrate in accordance with a mask, which in turn functions to define one or more microneedle features (e.g., body, base, tip, channel, reservoir, needle outline, etc.) in a future etching step. Additionally or alternatively, S230 can function to define one or more temporary features (e.g., sacrificial walls) of the microneedle device, wherein the temporary features are defined in a first future etching step and subsequently removed in a second future etching step.

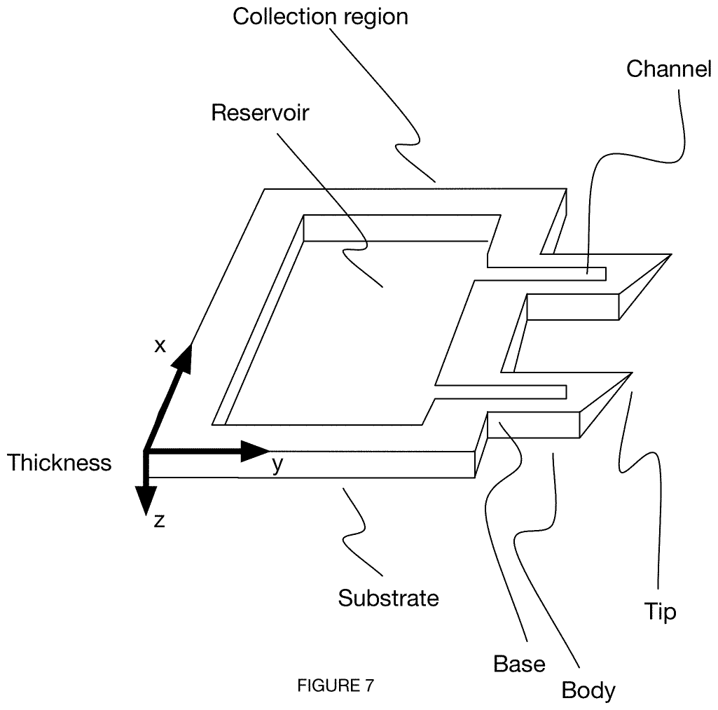

The substrate with photoresist is preferably patterned through selectively exposing the photoresist to light using a mask (e.g., photomask), wherein the mask defines a set of projected microneedle device features, the projected microneedle device features corresponding to features of the microneedle device at a projection aligned with a broad surface of the substrate (e.g., projection at a frontal plane of the needle, projection at a medial plane of the needle, etc.). Upon exposure to light, the features having projections defined by the mask become patterned into the photoresist. The set of projected features preferably include a needle cross section (having a projected needle length and a projected tip radius), and can optionally include one or more temporary features, such as one or more sacrificial walls (e.g., as shown in FIG. 3, as shown in FIG. 5), but can additionally or alternatively include other microneedle features (e.g., multiple needle outlines, multiple needle tips, needle diameters, channels, reservoirs, etc.), layout features (e.g., spacing between microneedles, connections between channels and reservoirs, etc.), temporary features (e.g., sacrificial features, sacrificial walls, etc.), or any other feature of the microneedle device.

The mask preferably includes a base with a set of projected features patterned on the base. The base preferably includes a transparent material (e.g., glass, plastic, etc.) but can additionally or alternatively be constructed from a semi-transparent material, non-transparent material, coated material (e.g., painted material), or any other suitable material. The set of projected features are preferably patterned on the base with chromium, but can additionally or alternatively be patterned with ink, film, or any other suitable material. The mask can be a positive mask, wherein the patterned projected features correspond to microneedle features to be retained during an etching process, or a negative mask, wherein the patterned projected features correspond to microneedle features to be removed during an etching process.

In one variation, the mask is a positive mask, wherein the patterned projected features include a microneedle cross section and, optionally, a sacrificial wall.

In a second variation, the mask is a negative mask, wherein the patterned projected features include a channel and a reservoir. However, the mask can define any suitable microneedle feature.

The patterned features can be substantially the same size as the corresponding resulting microneedle device features (e.g., length of patterned microneedle is the same as a resulting microneedle length). Additionally or alternatively, any or all of the patterned features can be overdrawn (e.g., patterned needle length 20 microns greater than desired needle length, patterned needle tip radius 5 microns greater than desired tip radius, etc.), which can function to account for extra etching or a post-etching treatment; underdrawn (e.g., patterned needle radius 1 micron smaller than desired needle radius, patterned needle length 10 microns smaller than desired needle length, etc.), which can function, for instance, to account for the addition of a coating.

Step S23o is preferably performed after S220, but can additionally or alternatively be performed at multiple times throughout the method (e.g., prior to performing each of a set of multiple etching steps), or at any other point during the method.

The substrate is preferably directly exposed to light at a broad surface of the substrate having a photoresist coating (e.g., light source aimed directly at broad surface). Additionally or alternatively, the light source can angled with respect to a surface of the substrate (e.g., oriented at a 45-degree angle with respect to a broad surface), multiple light sources can be aimed at the substrate (e.g., multiple light sources for multiple photoresist-coated surfaces), or the wafer and light source(s) can be arranged in any other suitable configuration.

The light source preferably includes ultraviolet (UV) light (e.g., wavelength between 10 nm and 400 nm), but can additionally or alternatively include light of a shorter wavelength (e.g., less than 10 nm), a longer wavelength (e.g., greater than 400 nm), or any other wavelength or combination of wavelengths. The light can be provided from the light source at any suitable power, dose, or intensity (e.g., 1800 milliJoules per centimeter squared).

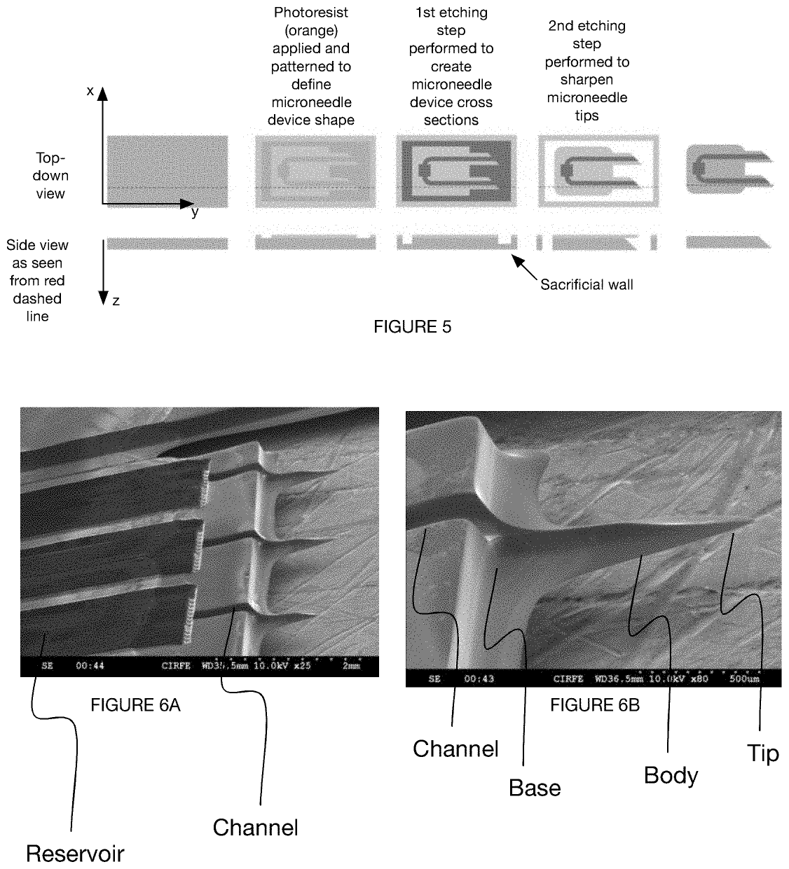

In a first variation, the mask includes a patterned needle cross section from a projection along a frontal or medial plane of the needle. The needle cross section can have any suitable shape, such as a trapezoid (e.g., with all right angles, no right angles, with some right angles, etc.), pentagon (e.g., with one or more right angles, with no right angles, etc.), triangle (e.g., with a right angle, with no right angles, etc.), or any suitable shape. The shape of the needle cross section preferably corresponds to the shape of one or more end faces of a prismatic needle body, but can alternatively have fewer sides, more sides, or any other shape. In a first specific example (e.g., as shown in FIG. 4), the mask includes a quadrilateral patterned needle cross section. In a second specific example, the mask includes a pentagonal patterned needle cross section.

In some variations (e.g., as shown in FIG. 3), the photoresist is patterned to define a set of multiple projected needle features, such as a set of needle cross sections, sacrificial walls, channels, and a reservoir.

In some variations, the patterned needle cross sections are overdrawn in comparison to the desired needle cross section dimensions, which can account for removal of material in a second etching step. Alternatively, the patterned needle cross section can be substantially equal to the desired dimensions or be underdrawn.

Step S230 can additionally include any additional sub-processes, such as, but not limited to, applying a developer (e.g., AZ 400k 4:1 developer).

### 4.3 Method—Performing a First Etching Treatment S240

The method can include performing a first etching treatment S240, which functions to create a first structure based on the set of patterned features. The first structure is preferably altered in a second etching treatment described below to form a completed microneedle device (e.g., sacrificial walls removed, tip sharpened, etc.), but can alternatively form a completed microneedle device without further modification. The first structure preferably includes a longitudinal needle cross-section formed from the removal of material adjacent to a patterned projected needle cross section and a sacrificial wall cross section formed from the removal of material adjacent to a patterned sacrificial wall cross section, but can additionally or alternatively include any other suitable feature or cross section.

The first etching treatment is preferably performed after photoresist has been applied and patterned (e.g., with a mask), but can additionally or alternatively be performed multiple times throughout the method, after a supplementary step (e.g., baking the substrate, curing the photoresist, etc.), or at any other time during the method.

The first etching treatment is preferably performed through anisotropic etching (e.g., Bosch process deep reactive ion etching (DRIE), cryogenic DRIE, etc.), further preferably through DRIE but can additionally or alternatively be performed through any other etching process. The first etching treatment preferably removes material of the substrate along a direction normal to a broad surface of the substrate, such as along the z-direction in FIG. 3. Additionally or alternatively, the etching can remove material in a direction parallel to a broad surface of the substrate or in any other suitable direction. The first etching preferably removes material of the substrate to a predetermined depth through any suitable number of cycles having any suitable running time. The first etching preferably etches to a depth of at least 10 microns, but can additionally or alternatively reach a depth between 10 and 20 microns, between 10 and 50 microns, between 2 microns and 0.5 mm, greater than 0.5 mm, or any other suitable depth. The first etching can be performed at any suitable etch rate such as: 1 micron/minute, less than or equal to 10 microns/minute, less than or equal to 20 microns/minute, between 10 and 20 microns/minute, greater than 20 microns/minute, or any other suitable etch rate.

The first etching can be performed cryogenically, through pulsed etching, continuous etching, time-multiplexed etching, and/or any other suitable form of etching. The first etching can be performed dry (e.g., with sulfurhexafluoride (SF6) plasma, nitrogen trifluoride, dichlorodifluoromethane, etc.) but can additionally or alternatively be performed wet (e.g., with potassium hydroxide/isopropyl alcohol, ethylenediamine pyrocatechol, tetramethylammonium hydroxide, hydrofluoric/nitric acid, etc.), or with any type and combination of etchants. The first etching treatment can further additionally or alternatively include isotropic etching.

The first etching step is preferably performed to a depth less than the thickness of the substrate, which allows for the formation of one or more connected sacrificial features (e.g., sacrificial walls). The substrate used is preferably thicker than the desired microneedle thickness (e.g., to accommodate the sacrificial material), but can alternatively have the same thickness as the desired microneedle. To achieve this, the first etching step can be continuously monitored, checked at regular or random intervals of time, stopped after a predetermined amount of time has passed, or performed in any other suitable way.

The parameters of any sacrificial features (e.g., thickness, length, width, volume, mass, distance from a needle cross section, etc.) can be determined based on a desired needle sharpness, wherein the needle sharpness is preferably at least partially formed in a second etching step but can alternatively be fully formed or not formed at all during a second etching step. In a first variation, the sacrificial feature parameters are calculated from any or all of: an isotropic etch rate, etchant mass transport rate though the material (e.g., silicon) of the substrate (e.g., estimated mass transport gradients), needle sharpness (e.g., tip radius), or any other suitable parameter.

In one variation, the length of a sacrificial wall and its distance from a needle tip is determined based on the mass transport properties of the substrate material (e.g., silicon) and a desired sharpness of the needle tip.

The first etching step can include any additional sub-processes, such as, but not limited to: passivation, reapplication of photoresist, polymer deposition, multiple etching steps with any number of etchant types, drying, curing, or any other sub-process.

In one variation in which the first etching step is performed through DRIE etching, Step S40 can include alternating etching (e.g., with fluorine-based gas such as SF6 gas) with polymer deposition (e.g., with gas such as C4F8).

In a second variation, DRIE etching is implemented in multiple blocks of cycles. In a specific example, for instance, 300 cycles of DRIE (e.g., through a Bosch process) are applied to etch to a first depth in the substrate (e.g., depth of 100 microns), and then 600 cycles of DRIE (e.g., through a Bosch process) are applied to etch to a second depth (e.g., 200 microns, 300 microns, etc.). Preferably, a baking step (e.g., at 250 degrees Celsius for 30 minutes) is performed after the second block of cycles to harden any remaining photoresist, but additionally or alternatively, a baking step can be performed between blocks of cycles. In some examples, the photoresist is partially or fully removed during or after the DRIE process.

### 4.4 Method—Perform a Second Etching Treatment S250

The method can include performing a second etching treatment S250, which functions to create a second structure from the first structure, the second structure having one or more needle features along the thickness of the needle (e.g., along the y-direction in FIG. 3). These needle features (e.g., undercuts) function to sharpen the needle but can additionally or alternatively function to change the shape of a needle end face (e.g., reduce the number of sides of the end face of a prism), shorten a needle of the first structure, reduce the overall diameter of a needle of the first structure, remove one or more sacrificial features of the first structure, or perform any other suitable function.

The second etching treatment is preferably performed after the first etching treatment S240 but can additionally or alternatively be performed during the first etching treatment, in lieu of the first etching treatment, prior to the first etching treatment (e.g., performed in reverse order with the first etching treatment), multiple times throughout the method, or at any other point during the method.

The second etching treatment is preferably performed through isotropic etching but can additionally or alternatively be performed through anisotropic etching. The second etching treatment preferably removes material of the substrate along a direction normal to a thickness of the substrate (e.g., along y-axis as shown in FIG. 4), thereby creating needle features along the needle thickness (e.g., undercut). Additionally or alternatively, the etching can remove material in a direction normal to a broad surface of the substrate, along a second direction normal to a thickness of the substrate (e.g., along x-axis), or in any other suitable direction.

The second etching can be performed cryogenically, through pulsed etching, continuous etching, time-multiplexed etching, and/or any other suitable form of etching. The second etching can be performed dry (e.g., with sulfurhexafluoride (SF6) plasma, nitrogen trifluoride, dichlorodifluoromethane, etc.), wet (e.g., with potassium hydroxide/isopropyl alcohol, ethylenediamine pyrocatechol, tetramethylammonium hydroxide, hydrofluoric/nitric acid, etc.), or with any type and combination of etchants. The photoresist can remain after the second etching (e.g., isotropic dry etching) but can alternatively be partially or fully removed during or prior to the second etching (e.g., isotropic wet etching). The first etching treatment can further additionally or alternatively include anisotropic etching.

In some variants, the second etching is performed through a “bottom-up” etching process, such as dry etching (e.g., as shown in FIG. 4), wherein the needle tip is sharpened in an inferior to superior direction (e.g., in a direction along the negative z axis in FIG. 4). In a specific example, a layer of photoresist is present in the second etching process, which prevents the substrate adjacent the photoresist (e.g., superior broad surface) from etching during the bottom-up etching process.

In other variants, a “top-down” etching process (e.g., as shown in FIGS. 8A-8G), such as wet etching (e.g., as shown in FIG. 4), is performed to sharpen the needle tip in a superior to inferior direction (e.g., in a direction along the positive z axis in FIG. 4). In a specific example, the needle tip is sharpened through a wet second etching step without a layer of photoresist present during the second etching.

The second etching step preferably leverages one or more sacrificial features (e.g., walls, borders, tabs, posts, etc.) bordering the needle tip of the first structure, such as, but not limited to, the sacrificial walls as shown in FIG. 3, to sharpen the needle tip. Additionally or alternatively, there can be any number of sacrificial features arranged proximal to the needle tip, proximal to the needle base, bordering an array of multiple needles, or otherwise arranged. Sacrificial features proximal the needle tip cause the needle tip to etch at different rates along its thickness due to mass transport principles, since the regions of the needle tip bordering substrate material (e.g., sacrificial wall) will etch slower than the regions not in contact with any substrate material. This creates a sloped profile along the needle tip thickness (e.g., based on a truncation plane as previously described), which thereby sharpens the needle. The sloped profile can be straight (e.g., as shown in FIG. 4) and sloped at any suitable angle (e.g., 45 degrees with respect to the y-axis, 30 degrees, less than 30 degrees, between 30 degrees and 60 degrees, greater than 60 degrees, etc.), curved (e.g., concave, convex, etc.), jagged, or have any other suitable profile.

The second etching preferably removes material of the substrate to a variable depth as measured normal to a substrate thickness, thereby creating a sloped profile, but can alternatively remove material to a uniform depth. The depth can have any suitable value such as 10 microns, between 10 and 20 microns, between 10 and 50 microns, between 2 microns and 0.5 mm, greater than 0.5 mm, or any other suitable value. The second etching can be performed at any suitable etch rate at any thickness of the needle tip, such as 1 micron/minute at a first thickness and 2 microns/minute at a second thickness, less than or equal to 10 microns/minute at any thickness, less than or equal to 20 microns/minute at any thickness, between 10 and 20 microns/minute at any thickness, greater than 20 microns/minute at any thickness, or any other suitable etch rate.

The parameters of the second etching (e.g., etch rate, etch duration, temperature, humidity, etc.) are preferably configured to define a needle with a predetermined set of features as described previously and can be determined based on one or more desired dimensions (e.g., desired needle tip radius, desired needle length, desired needle thickness, etc.), one or more features of the substrate (e.g., oxidization rate, thickness, etc.), or any other suitable feature or desired feature. The resulting tip can have any suitable shape (e.g., prism, truncated prism, cone, pyramid, cylinder, etc.), with any suitable number of faces with any suitable number of sides. The tip can be held constant (e.g., stationary) during the second etching but can additionally or alternatively be modulated (e.g., dynamically moved according to a schedule), translated, rotated (e.g., to result in smooth sides of the needle tip, form a cone tip, etc.), or otherwise manipulated during the second etching treatment.

In some variations, the second etching step is performed with a mask. Alternatively or additionally, a mask (e.g., previously-applied photoresist) can be removed prior to S250. The mask can function to preclude or slow etchant ingress (e.g., to a non-tip needle feature), protect one or more features (e.g., needle features, sacrificial features, etc.), or perform any other suitable function. The mask can be constructed from any of the materials described previously (e.g., be the previously-applied photoresist layer), include simple mechanical barrier (e.g., include only a base), include a simple optical barrier (e.g., tinted film), or any other suitable material. The second etching step can further include applying photoresist (e.g., if a pattern is desired to be etched into the thickness). The mask can be applied to the tip side or elsewhere on or within the second structure. The mask is preferably applied prior to the second etching but can additionally or alternatively be applied prior to the first etching, during the first etching, or at any other point during the method.

The second etching step can include any additional sub-processes, such as, but not limited to: passivation, reapplication of photoresist, multiple etching steps with any number of etchant types, drying, curing, or any other sub-process.

In one variation, the second etching is performed through dry etching (e.g., SF6 plasma etching). In a specific example (e.g., as shown in FIG. 4), the tip cross section as viewed from a side (e.g., viewed from direction normal to thickness) slopes inward as a result of the second etching.

In a second variation, the second etching is performed through wet etching (e.g., hydrofluoric/nitric acid etching). In a specific example (e.g., as shown in FIG. 4), the tip cross section as viewed from a side (e.g., viewed from direction normal to thickness) slopes outward as a result of the second etching.

In a third variation of the second etching step, the second etching step is at least partially performed through wet etching (e.g., using nitric acid and hydrofluoric acid), wherein the etch rate of the substrate (e.g., silicon) is limited to a rate of oxide removal of the substrate. In a specific example using a mixture of nitric acid and hydrofluoric acid to etch a silicon wafer substrate, wherein the nitric acid first oxidizes silicon into silicon dioxide and the hydrofluoric acid then etches away the silicon dioxide, the etching rate of the silicon is based on (e.g., limited to) the rate of oxide removal.

In a fourth variation, for substrate initially having double-layered photoresist, SF6 etching can be performed for 80 min. In a specific example, the etching is performed in a machine configured for DRIE etching.

In a fifth variation, for a substrate initially having single-layered photoresist, purely isotropic etching with SF6 can be performed first (e.g., for 30 min at a flow rate of 160 sccm) followed by wet etching (e.g., for 60 min).

### 4.6 Method—Supplementary Steps

The method 200 can optionally include any number of additional steps, such as pretreating a substrate S210, which functions to prepare the substrate (e.g., creating a clean and dry surface, enhancing adhesion with a photoresist, creating a substrate of predetermined shape and size, etc.) for any or all of the remaining steps of the method. Step S210 is preferably performed prior to S220, but can additionally or alternatively be performed prior to any step of the method, at multiple times throughout the method, or at any other point. Pretreating a substrate can include any or all of: baking the substrate (e.g., at 200 degrees Celsius for 3 minutes), storing the substrate in a desiccator, cleaning the substrate (e.g., with acetone, isopropanol, ethanol, etc.), or any other suitable pretreatment. In one variation, Step S210 includes cleaning the wafer by immersion in hydrofluoric acid followed by a solvent clean, deionized (DI) water rinse, and a blow dry.

Additionally or alternatively, the method can include any number of sub-processes to prepare the microneedle device for use S260, such as, but not limited to: removing photoresist (e.g., during one of the etching treatments, during a wet etching treatment, through an oxygen plasma treatment, etc.), curing photoresist, assembling multiple microneedle devices (e.g., rows of microneedles) together (e.g., to form a microneedle patch), attaching one or more microneedle devices to a base or housing or other packaging, adding a biocompatible material (e.g., biocompatible coating) to the microneedle device, adding a wicking material (e.g., paper, fabric, etc.), or any other suitable sub-process. Step S260 is preferably performed after step S250 but can additionally or alternatively be performed at any other point during the method.

The method 200 can additionally or alternatively include any other suitable steps performed in any suitable order.

In a variation of the method 200, the method includes coating a surface of a silicon wafer with photoresist; layering a mask on a broad surface of the silicon wafer, the mask defining a set of patterned features including multiple needle cross sections, a sacrificial border, a set of channels, and a reservoir; exposing the mask to UV light; performing deep reactive ion etching to create a first structure defining a set of microneedles with etched channels, an etched reservoir, and partially-etched sacrificial walls; performing isotropic etching to create undercuts at the needle tips thereby sharpening the needles.

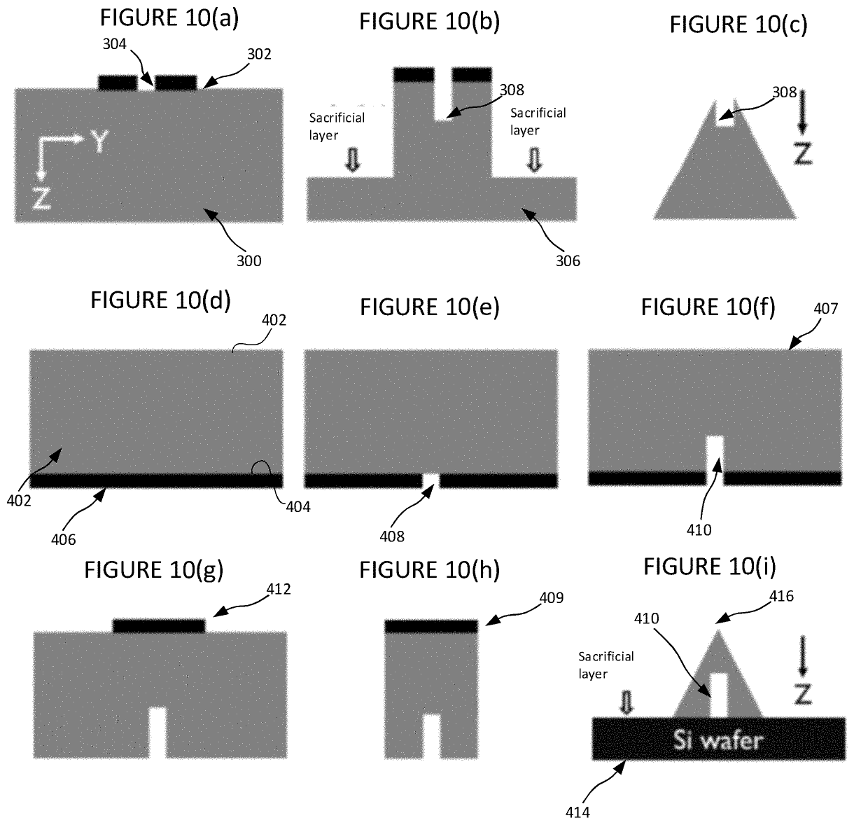

As it may be recognized by a skilled person in the art, in embodiments where the second etching is performed through an isotropic wet etching, the open channels and reservoir formed on the first structure from the first etching are also exposed to the etchant of the second etching and at least partially etched away as shown in FIGS. 10(a) to 10(c). In particular, FIG. 10(a) shows an exemplary substrate 300 spin-coated with a photoresist 302. Then the photoresist 302 is patterned using any one suitable methods, such as photolithography, to create one or more features of a microneedle. In the non-limiting exemplary embodiment shown in FIG. 10(a), a channel opening 304 is formed in the patterned photoresist 302. Then the first etching is applied to the substrate 100 allowing the DRIE process to simultaneously etch portions of the substrate not protected by the patterned photoresist 302. The first structure resulting from the first etching is shown in FIG. 10(b). As shown, portions of the substrate 300 directly beneath the patterned photoresist 302 are preserved. The adjacent portions of the substrate 300 are etched away to an etching depth that is less than the total thickness of the substrate 300 so as to create a sacrificial feature in the form of a sacrificial layer 306. Channel 308 is etched to a depth less than the etching depth due to aspect ratio dependent etching (ARDE) principles, where the etching rate rapidly decrease with the aspect-ratio of the etched structures. Thus, the depth of channel 308 may be controlled by the dimensions of the channel opening 304 formed in the photoresist 302. Then isotropic etching, such as a wet chemical etching, is utilized to, among other functions, sharpen the needle shaft structure in the presence of the sacrificial layer 306. In an example embodiment, the isotropic wet etching uses an etchant composed of nitric acid (HNO₃) and hydrofluoric acid (HF) at a volume ratio of 19:1. In this particularly example embodiment, the HNO₃ oxidizes the silicon into silicon dioxide (SiO₂), which is subsequently etched away by HF. As a person skilled in the art may appreciate the etch rate of silicon is limited by the rate of oxide removal. Thus, HF diffusion to the silicon surface is the rate-limiting factor. As the etching of the sacrificial feature depletes HF in the surrounding etchant solution, the etching rate is slowed compared to that near the top of the needle shaft. Thus, over time, the top of the needle shaft is sharpened while the base is mostly maintained with little or no sacrificial feature left as shown in FIG. 10(c). As it may be evident from FIG. 10(c), the depth of the channel 308 after the second etching is less than of the channel 308 after the first etching as shown in FIG. 10(b).

Accordingly, in another example embodiment of the present disclosure, there is provided a double-sided processing method 400 for manufacturing an in-plane microneedle that may advantageously minimize unintended etching of the channels during the second etching processing. An example embodiment of method 400 is shown in FIGS. 10(d) to 10(i).

Specifically, as shown in FIG. 10(d) there is provided a substrate 402 with a first broad surface 404 and an opposing second broad surface 406. At least one layer of photoresist is applied to the second surface 406 using any suitable methods as disclosed above. Then the photoresist 406 is patterned using any of the previously mentioned suitable methods to define a microfluidic network that comprises at least one channel opening 408 as shown in FIG. 10(e). Then a first etching, such as Bosch DRIE process or any other suitable process, is carried out on the substrate 402 to derive a first structure 407 with an etched microfluidic network. As shown in FIG. 10(f) a channel 410 of the microfluidic network is etched from the second surface 406 of the substrate 402 in the first structure 407. The channel depth of channel 410 may be similarly based on ARDE principles as disclosed above. Then at least one photoresist layer 412 may be applied to the first broad surface 402 and patterned to define one or more features of a microneedle as shown in FIG. 10(g). A second etching treatment, such as a Bosch DRIE process, is applied to the first structure 407 from the first surface 402. As shown in FIG. 10(h) the first structure 407 is etched through during the second etching treatment to define a second structure 407 as a needle shaft of the microneedle while channel 410 is preserved on the second surface 404. In the example embodiment shown in the figures, the photoresist 412 on the first surface 402 may be patterned so as to align with the channel 410 formed on the second surface 404. Such alignment may advantageously enable more accurate placement of the channels with offsets from the axis of the needle shaft, positioned away from an apex of the sharpened tip, which may reduce the tissue coring of the channels. Any photoresist 412 on the first surface 402 may be removed using any suitable cleaning methods. A clean silicon wafer 414 is attached to the second structure 409 from the second surface 404 by any suitable methods such that channel 410 is in direct contact with a polished side of the wafer 414 to minimize the space between them and thus the volume of the wet etching solution that may enter. In some example embodiments, wafer 414 is attached the second structure 409 using a Kapton® tape. As understood in the art, Kapton® tape is a polyimide film with a Silicon adhesive. In this second structure, at least wafer 414 acts as a sacrificial feature. A third etching process, such as an isotropic wet etching, is applied to the second structure to derive a third structure which comprises at least a sharpened tip 416 of the microneedle while channel 410 remains intact with minimal etching during the third etching process as shown in FIG. 10(i). The sacrificial feature, including at least the wafer 414, can consume significant amount of etchant species, such as HF in the example embodiment mentioned above, resulting in the depletion of such etchant species at the surface of the sacrificial structure, since the depleted etchant species could only be transported from the bulk solution via the slow diffusion for a static solution. However, the concentration of the same etchant species near the top of the needle, away from the sacrificial feature, is higher because it is closer to the bulk solution. As a consequence, the introduction of the sacrificial feature may enable a buildup of a concentration gradient of the etching species, resulting in faster etching rate near the top of the microneedle shaft than the bottom, which is closer to the sacrificial feature, thereby forming a wedge-shaped structure with a sharpened tip tapering smoothly to the base of the needle shaft.

Although omitted for conciseness, the example embodiments include every combination and permutation of the various system components, method processes, variations, and examples.

As a person skilled in the field will recognize from the previous detailed description and figures, modifications and changes can be made to the variations and examples of the invention without departing from the scope of the invention.

1. A method of fabricating a needle, the method comprising:
providing a semiconductor substrate;
applying a first photoresist to a first surface of the substrate;
patterning the first photoresist to define a first etching pattern;
performing a first etching treatment to the substrate, the first etching pattern causing the substrate to be etched into a first structure, the first structure including at least one sacrificial feature; and
performing a second etching treatment on the first structure to etch the first structure into a second structure, wherein the at least one sacrificial feature is at least partially etched away during the second etching treatment causing one or more features of the needle to be etched into the second structure.

2. The method of claim 1 wherein the method is an in-plane method of fabricating the needle.

3. The method of claim 1, further comprising pre-treating the substrate, which includes baking the substrate, cleaning the substrate, creating a clean and dry first surface, enhancing adhesion with the first photoresist, cleaning the substrate by immersion in hydrofluoric acid, cleaning by a solvent, rinsing by deionized water, or blow drying.

4. The method of claim 1, further comprising one or more of removing the first photoresist, curing the first photoresist, assembling multiple needles together, adding a biocompatible material to the needle, or adding a wicking material.

5. The method of claim 1, wherein the applying includes applying a single layer of the first photoresist, or a double layer of the first photoresist.

6. The method of claim 1, wherein
the patterning includes defining a channel opening with a width in the first photoresist, the first etching treatment including etching a channel into the first structure by aspect ratio dependent etching (ARDE), a depth of the channel is defined by the width of the channel opening.

7. The method of claim 6, wherein the at least partial etching of the at least one sacrificial feature during the second etching treatment causes the one or more needle feature to include a sharpened needle tip.

8. The method of claim 1, wherein the one or more features of the needle include the shape of a needle end face, a length of the needle, and an overall diameter of the needle.

9. The method of claim 1, wherein the first etching treatment comprises deep reactive ion etching (DRIE).

10. The method of claim 1, wherein the first etching treatment is performed so that portions of the substrate adjacent to the patterned first photoresist is etched to a depth less than a total depth of the substrate thereby defining the at least one sacrificial feature.

11. The method of claim 1, wherein the one or more features of the needle include at least one needle cross section, etching away the at least one sacrificial feature during the second etching treatment causes a variation in an etching rate along a lengthwise direction of the at least one needle cross section such that a first end of the at least one needle cross section is etched into a sharpened needle tip.

12. The method of claim 1, wherein the second etching treatment comprises isotropic wet etching.

13. The method of claim 1, wherein the second etching treatment further comprises removing portions of the first structure to a variable depth thereby creating a sloped profile.

14. The method of claim 1, wherein the substrate is a low resistivity, p-type, single side-polished silicon wafer with a predetermined crystal orientation.

15. The method of claim 1, wherein a length of the needle is between 1 mm to 2 mm.

16. The method of claim 1, further comprising:
applying a second photoresist to a second surface of the substrate,
patterning the second photoresist to define a second etching pattern, the second etching pattern defines at least one fluidic channel of the needle,
performing a third etching treatment to the substrate, the second etching pattern causing the at least one fluidic channel to be etched into the second surface of the substrate;
attaching an additional sacrificial feature to the second surface of the second structure such that the at least one fluidic channel is covered by the additional sacrificial feature; and
wherein the additional sacrificial feature that is at least partially etched away during the second etching treatment minimizes or eliminates any etching to the at least one fluidic channel during the second etching treatment.

17. (canceled)

18. The method of claim 16, further comprising removing the second photoresist after the third etching treatment.

19. The method of claim 16, wherein the at least one fluidic channel defines a microfluidic network.

20. The method of claim 16, wherein the third etching treatment comprises DRIE.

21. The method of claim 16, wherein the additional sacrificial feature comprises a silicon wafer.

22. The method of claim 16, wherein the additional sacrificial feature is attached to the second surface of the substrate with tape comprising a polyimide film with a silicon adhesive.

23. The method of claim 16, wherein the at least one channel extends through the needle tip and positioned away from an apex of the tip.

24. The method of claim 16, wherein the at least one channel extends through the tip offset at an axis away from a longitudinal axis defined by the tip

25. The method of claim 1, further comprising:
applying a second photoresist to a second surface of the substrate,
patterning the second photoresist to define a second etching pattern, the second etching pattern defines at least one fluidic channel of the needle, and
performing a third etching treatment to the substrate, the second etching pattern causing the at least one fluidic channel to be etched into the second surface of the substrate.

26. The method of claim 1, wherein the needle is a microneedle, wherein the tip has a radius within micron range.

27. The method of claim 1, wherein the tip has a radius less than or greater than micron range.

28. A needle for reaching and sampling interstitial fluid of a user produced by the method of claim 1.
